(19) United States
(12) Patent Application Publication
(10) Pub. No.: US 2006/0247631 A1
(43) Pub. Date: Nov. 2, 2006

(54) Spinal pedicle screw assembly

(76) Inventors: Sae Young Ahn, Pyeongtaig City (KR); Jae Kwan Lim, Pyeongtaig City (KR); Jong Myeon Choi, Suwon City (KR); Hoon Lee, Jeju City (KR); Chang Soo Lim, Suwon City (KR); Seo Kon Kim, Pyeongtaig City (KR); Se Ryeong Kim, Seoul (KR); Hoon Kim, Pyeongtaig City (KR)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 11/254,055

(22) Filed: Oct. 19, 2005

(30) Foreign Application Priority Data
Apr. 27, 2005 (KR) 10-2005-0034961
Jun. 13, 2005 (KR) 10-2005-0050435

(51) Int. Cl. A61F 2/30

(52) U.S. Cl. 606/61

(57) ABSTRACT

Disclosed is a spinal pedicle screw assembly including a spinal pedicle screw cooperating with a spine rod for fixing a spine, which includes: a head-coupling element having an upper and a lower end, a rod-receiving channel opening toward the upper end for receiving the spine rod, female screw threads formed on the inside of the rod-receiving channel, and a connecting bore of a given size extending through the lower end, the connecting bore having an upper end toward the rod-receiving channel and an opposite lower end; a screw rod having an upper and a lower end, a spherical head integrally formed with the upper end thereof for being inserted into the connection bore, and male screw threads formed on the periphery of the screw rod between the spherical head and lower end thereof, the screw rod being fixedly inserted into the pedicle by means of the male screw threads thereof; a tightening screw having a periphery formed with male screw threads for engaging the female screw threads of the rod-receiving channel so as to fix the spine rod in the head-coupling element; and a final fixing screw threadedly engaged by the connecting bore for preventing the spherical head from disconnecting from the head-coupling element.

### BACKGROUND OF THE INVENTION

1. Field of the Invention

The present invention relates to a spinal pedicle screw assembly including a spinal pedicle screw that cooperates with a spine rod for stabilizing a spine. In this technical field, the term spine rod usually means an orthopedic rod used in the spinal fixation technique for immobilizing the spine.

2. Description of the Related Art

The spinal column is a highly complex system of bones and connective tissues that provides support for the body and protects the delicate spinal cord and nerve root. The spinal column includes a series of vertebrae stacked one atop the other, each vertebral body including an inner or central portion of relatively weak cancellous bone and an outer portion of relatively strong cortical bone.

The spinal column disorders are usually cured in two ways, one of which is an indirect method of physical therapy, and the other is a direct method using an artificial fixation device that is implanted in the spine so as to align and immobilize the vertebrae of the spine. The physical therapy is usually used for curing a slightly disordered spinal column, but the artificial fixation device will be used for curing a seriously disordered spinal column wherein the cervical vertebrae, thoracic vertebrae, lumbar vertebrae, sacrum or intervertebral discs are seriously impaired.

One spinal fixation technique involves immobilizing the spine by using the spine rods arranged generally parallel to the spine. To this end is exposed the spine posteriorly and the spinal pedicle screws fastened to the pedicles of the appropriate vertebrae. The pedicle screws are generally placed two per vertebra and serve as anchor points for the spine rods. The aligning influence of the rods forces the spine to conform to a more desirable shape.

The spinal pedicle screw assembly usually includes a screw rod fixedly inserted into the pedicle, and a head-coupling element connected to the screw rod. Conventionally, there are two kinds of pedicle screws according to the method of connecting the screw rod and head-coupling element. One kind integrates the screw rod and head-coupling element into a single unit, usually called “single-axis spinal pedicle screw assembly”, and the other rotatably connects the head-coupling element with the screw rod, usually called “multi-axis spinal pedicle screw assembly”. The multi-axis spinal pedicle screw assembly generally comprises a screw rod with a spherical head formed at one end thereof, a head-coupling element having an upper and a lower end, and a screw member, wherein the screw rod is undetachably inserted into the lower end of the head-coupling element, and the screw member externally locks the head-coupling element from the upper end thereof so as to fix the spine rod by pressing, thereby establishing multiple axes. However, such spinal pedicle screw assembly suffers such drawback that an external force may loosen the externally locking screw member, thus resulting in an insufficient locking and restraining force to fix a disordered spine.

In addition, the recent spinal fixation technique requires a miniaturized spinal pedicle screw assembly. To meet this requirement, the head-coupling element is designed to have an internal female screw thread internally engaging a screw member to fix the spine rod, thereby preventing the diameter of the head-coupling element from increasing. However, this also cannot adequately compensate for an external force, so that the bodily motion causes the screw member to loosen from the internally engaging head-coupling element after a long time, thus producing a problem in the spinal fixation.

### SUMMARY OF THE INVENTION

It is an object of the present invention to provide a spinal pedicle screw assembly comprising a head-coupling element and a screw rod that prevents a spine rod from loosening even by a strong external force.

It is another object of the present invention to provide a spinal pedicle screw assembly comprising a head-coupling element and a screw rod that a strong frictional force is produced between the head-coupling element and the screw rod.

According to an aspect of the present invention, there is provided a spinal pedicle screw assembly including a spinal pedicle screw cooperating with a spine rod for fixing a spine, which comprises: a head-coupling element having an upper and a lower end, a rod-receiving channel opening toward the upper end for receiving the spine rod, female screw threads formed on the inside of the rod-receiving channel, and a connecting bore of a given size extending through the lower end, the connecting bore having an upper end toward the rod-receiving channel and an opposite lower end; a screw rod having an upper and a lower end, a spherical head integrally formed with the upper end thereof for being inserted into the connection bore, and male screw threads formed on the periphery of the screw rod between the spherical head and lower end thereof, the screw rod being fixedly inserted into the pedicle by means of the male screw threads thereof; a tightening screw having a periphery formed with male screw threads for engaging the female screw threads of the rod-receiving channel so as to fix the spine rod in the head-coupling element; and a final fixing screw threadedly engaged by the connecting bore for preventing the spherical head from disconnecting from the head-coupling element.

Preferably, a restraining plate fixedly is positioned between the upper end of the connecting bore and the spherical head for restraining the spherical head from moving. The cut between the female screw threads of the rod-receiving channel are tapered towards the central axis of the head-coupling element. The upper end portion of the connecting bore is cut to form a seat for stopping the restraining plate. The restraining plate has a lower surface provided with a head receiving depression matching the curvature of the spherical head. The spherical head has a flat top. The flat top is provided with a central wrench depression of a given shape. The surface of the spherical head is knurled. The thickness of the male screw threads formed on the periphery of the screw rod is gradually increased towards the spherical head. The tightening screw has a top provided with a central wrench depression of a given form. The final fixing screw has a central through hole for receiving the spherical head, the inside of the central through hole being provided with a lower part shaped to match the curvature of the spherical head.

According to another aspect of the present invention, there is provided a spinal pedicle screw assembly including a spinal pedicle screw cooperating with a spine rod for fixing a spine, which comprises: a head-coupling element having an upper and a lower end, a rod-receiving channel opening toward the upper end for receiving the spine rod, and female screw threads formed on the inside of the rod-receiving channel; a screw rod integrally formed with the lower end of the head-coupling element and having a periphery provided with male screw threads, the screw rod being fixedly inserted into the pedicle by means of the male screw threads thereof; and a tightening screw having a periphery formed with male screw threads for engaging the female screw threads of the rod-receiving channel so as to fix the spine rod in the head-coupling element.

Preferably, the cut between the female screw threads of the rod-receiving channel are tapered towards the central axis of the head-coupling element. The thickness of the male screw threads formed on the periphery of the screw rod is gradually increased towards the head-coupling element. The tightening screw has a top provided with a central wrench depression of a given shape.

The present invention will now be described more specifically with reference to the drawings attached only by way of example.

### BRIEF DESCRIPTION OF THE ATTACHED DRAWINGS

FIG. 1 is a schematic perspective view for illustrating the shape of a spinal pedicle screw assembly according to an embodiment of the present invention;

FIG. 2 is a partially cross-sectional view for illustrating a spine rod fixedly positioned between the head of a screw rod and a tightening screw internally engaged by the head-coupling element of a spinal pedicle screw assembly as shown in FIG. 1;

FIG. 3 is an exploded view of FIG. 2 for more clearly illustrating the relationship between the spinal pedicle screw and the spine rod;

FIG. 4 is a perspective view for illustrating the shape of the head-coupling element of a spinal pedicle screw assembly according to an embodiment of the present invention;

FIG. 5 is a cross-sectional perspective view taken along line A-A′ of FIG. 4 for illustrating the cut between the female screw threads of the head-coupling element;

FIG. 6 is a perspective view of the head-coupling element of FIG. 4 for illustrating the connecting bore;

FIG. 7 shows a schematic perspective view for illustrating the shape of the screw rod of a spinal pedicle screw assembly according to an embodiment of the present invention and a cross-sectional view thereof;

FIG. 8 is a perspective view of the tightening screw of a spinal pedicle screw assembly according to an embodiment of the present invention;

FIG. 9 is a cross-sectional perspective view taken along lines 8A-8A″ and 8A″-8A′ of FIG. 8 for illustrating the cut between the female screw threads of the head-coupling element;

FIG. 10 is a perspective view for illustrating the shape of a spinal pedicle screw assembly according to another embodiment of the present invention;

FIG. 11 is a partially cross-sectional view for illustrating a spine rod fixedly positioned between the head-coupling element of a spinal pedicle screw assembly as shown in FIG. 10 and a tightening screw internally engaged by the head-coupling element; and

FIG. 12 is an exploded view of FIG. 11 for more clearly illustrating the relationship between the spinal pedicle screw and the spine rod.

### DETAILED DESCRIPTION OF THE PREFERRED EMBODIMENTS

Throughout descriptions in connection with the drawings, same reference numerals are used to represent same functional parts for convenience's sake. In addition, there are omitted detailed descriptions of the conventional parts not required to comprehend the technical concept of the present invention.

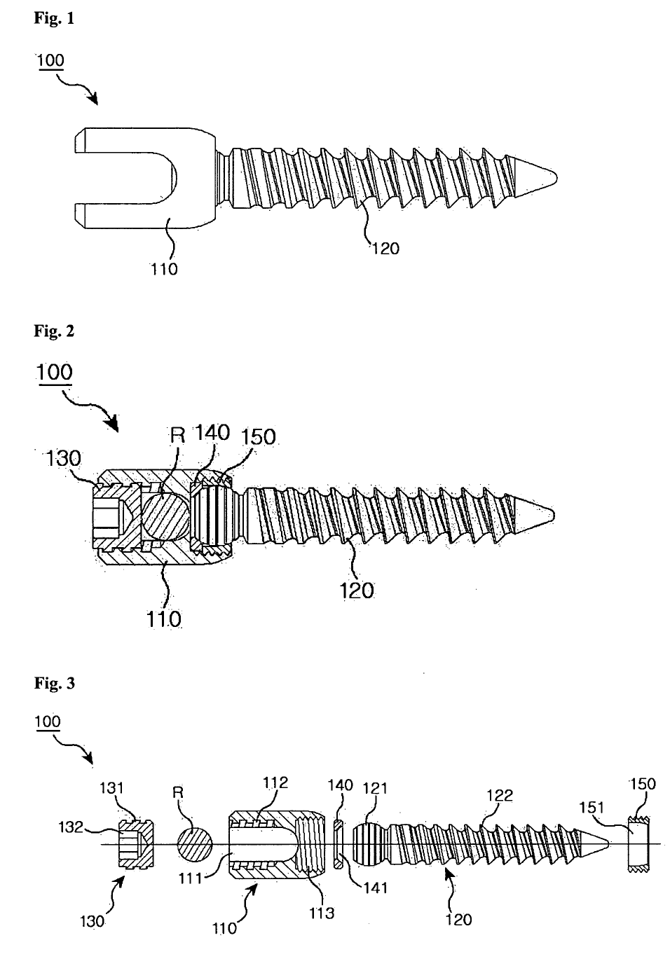

Referring to FIGS. 1 to 3, a spinal pedicle screw assembly 100 according to an embodiment of the present invention includes a head-coupling element 110 with an upper and a lower end for receiving a spine rod R, a screw rod 120 having an upper and a lower end and a spherical head 121 integrally formed with the upper end thereof, and a tightening screw 130 for fixing the spine rod R in the head-coupling element 110. The head-coupling element 110 has a rod-receiving channel 111 opening toward the upper end for receiving the spine rod R, female screw threads 112 formed on the inside of the rod-receiving channel 111, and a connecting bore 113 of a given size extending through the lower end. The spherical head 121 is inserted into the connecting bore 113. Preferably, a restraining plate 140 is fixedly positioned between the upper end of the connecting bore 113 and the spherical head 121 for restraining the spherical head from moving. Also provided is a final fixing screw 150 threadedly engaged by the connecting bore 113 for preventing the spherical head from disconnecting from the head-coupling element 110.

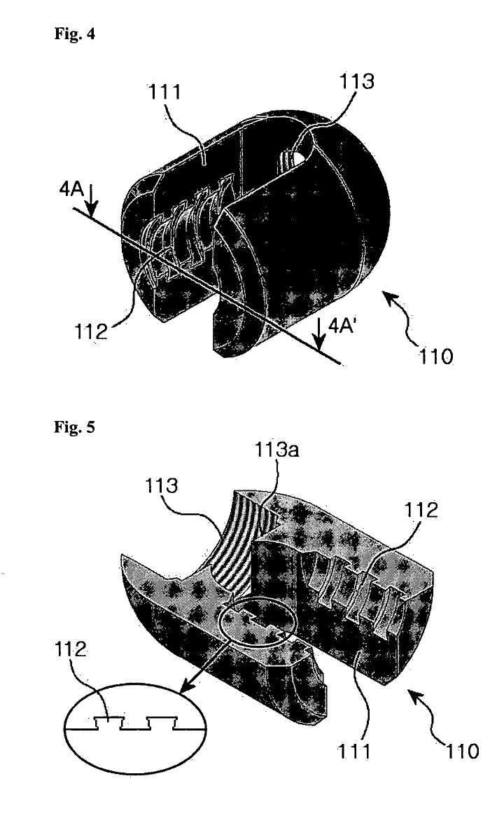

Referring to FIGS. 4 to 6, the head-coupling element 110 is shaped like a cup with a rounded bottom, having the U-shaped rod-receiving channel 111 cut through its side wall and the connecting bore 113 cut through its bottom.

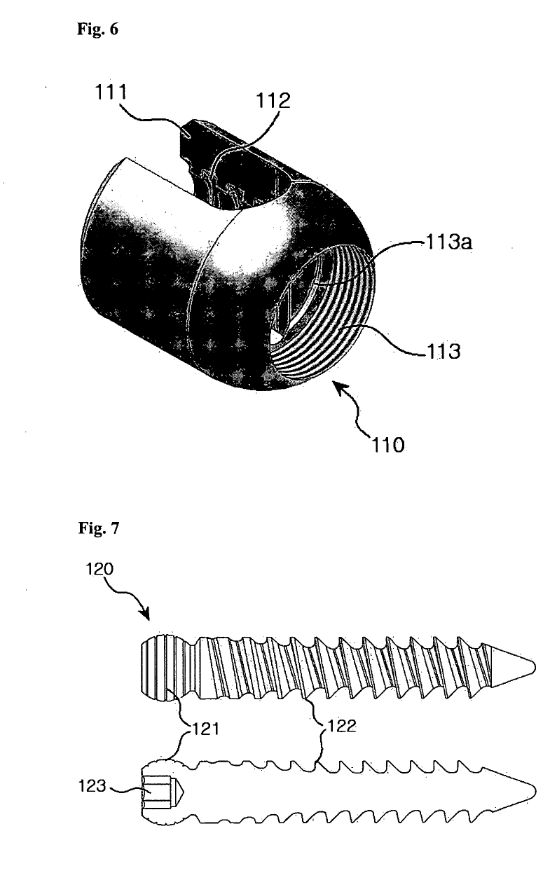

The internal width of the U-shaped rod-receiving channel 111 is designed to snugly receive the spine rod R so as to prevent it from laterally moving, and the female screw threads 112 are formed along the curvature of the inside thereof, tapering towards the periphery of the head-coupling element 110 (See the view indicated by a circle in FIG. 5). In other words, the cut between the female screw threads 112 are tapered towards the central axis of the head-coupling element 110. The central axis of the taper is perpendicular to the central axis of the head-coupling element 110. This taper maximizes the interlocking force between corresponding male screw threads 131 formed on the periphery of the tightening screw 130 and the female screw threads 112 of the rod-receiving channel 111, protecting the interlocking state from being adversely affected by a vertical external force.

The connecting bore 113 has a diameter greater than that of the rod-receiving channel 111, of a given size extending through the lower end. The connecting bore has an upper end toward the rod-receiving channel and an opposite lower end. The upper end of the connecting bore is cut to form a seat 113a shaped like a pair of wings for stopping the restraining plate 140. The inside wall of the connecting bore is provided with female screw threads to engage the final fixing screw 150.

Referring to FIG. 7, the spherical head 121 is integrally formed with the upper end of the screw rod 120. Male screw threads 122 are formed on the periphery of the screw rod between the spherical head and the lower end thereof. The screw rod 120 is turned by a separate turning device (not shown), fixedly inserted into the pedicle.

The spherical head 121 is cut to form a flat top, and has its surface knurled so as to maximize the frictional force with the restraining plate 140. The flat top is provided with a central wrench depression 123 of hexagonal or octagonal shape, whereby a separate turning device such as wrench is used to insert the screw rod 120 into the pedicle.

The screw rod 120 is tapered towards its lower end. More specifically, the lower end of the screw rod is cone-shaped to facilitate its insertion into the pedicle, and its diameter becomes largest at the upper end thereof. In addition, the thickness of the male screw threads 122 is gradually increased towards the spherical head 121, having maximum at the upper end, so as to maximize the final fixing force of the screw rod 120 inserted into the pedicle.

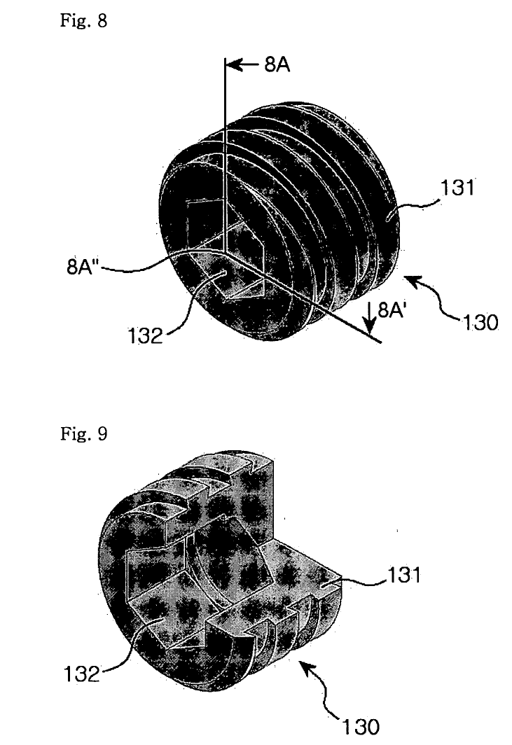

Referring to FIGS. 8 and 9, the tightening screw 130 has the periphery provided with the male screw threads 131 tapering towards the center of the head-coupling element 110 to engage the female screw threads 112, and a top provided with a hexagonal or octagonal wrench depression 132, whereby the tightening screw 130 is turned by a wrench (not shown), inserted into the rod-receiving channel 111 to fix the spine rod R in cooperation with the head-coupling element 110.

Referring again to FIGS. 2 and 3, the restraining plate 140 interposed between the upper end of the connecting bore 113 and the spherical head 121 has a lower surface provided with a head receiving depression 141 matching the curvature of the spherical head 121, thus serving to restrain the spherical head 121 from moving upon fixing the spine rod R.

The final fixing screw 150 has a central through hole 151 for receiving the spherical head 121. The inside of the central through hole 151 is provided with a lower part shaped to match the curvature of the spherical head 121 so as to prevent it from separating. The final fixing screw 150 has a periphery provided with male screw threads matching the female screw threads of the connecting bore 113. Thus, the final fixing screw 150 is threadedly engaged by the connecting bore 113.

More specifically describing the parts of the inventive spinal pedicle screw assembly with reference to FIGS. 2 and 3, the restraining plate 140 is inserted into the connecting bore 113, mounted on the seat 113a, and then the spherical head 121 closely fitted into the receiving depression 141, and the final fixing screw 150 threadedly engaged by the connecting bore 113 by receiving the screw rod 120, thereby preventing the spherical head 121 from separating.

The screw rod 120 of the spinal pedicle screw assembly 100 is turned by a wrench (not shown) engaging the wrench depression 123, fixedly inserted into a pedicle. Then, the spine rod R is inserted into the rod-receiving channel 111, over which the tightening screw 130 is turned by a wrench (not shown) to press the spine rod R towards the restraining plate 140 and spherical head 121, thereby maximizing the frictional contact force between them to restrain the screw rod 120 from moving.

FIG. 2 illustrates the spine rod R fixed in the spinal pedicle screw assembly 100 according to the present invention, wherein the female screw threads 112 of the head-coupling element 110 and the corresponding male screw threads 131 of the tightening screw 130 are tapered so as to prevent the spine rod R from loosening due to an external force exerted towards the body portion containing the spine rod. In addition, the knurled spherical head 121 closely contacts the restraining plate 140 with a considerably large frictional resistance, thus preventing the spine rod from slipping.

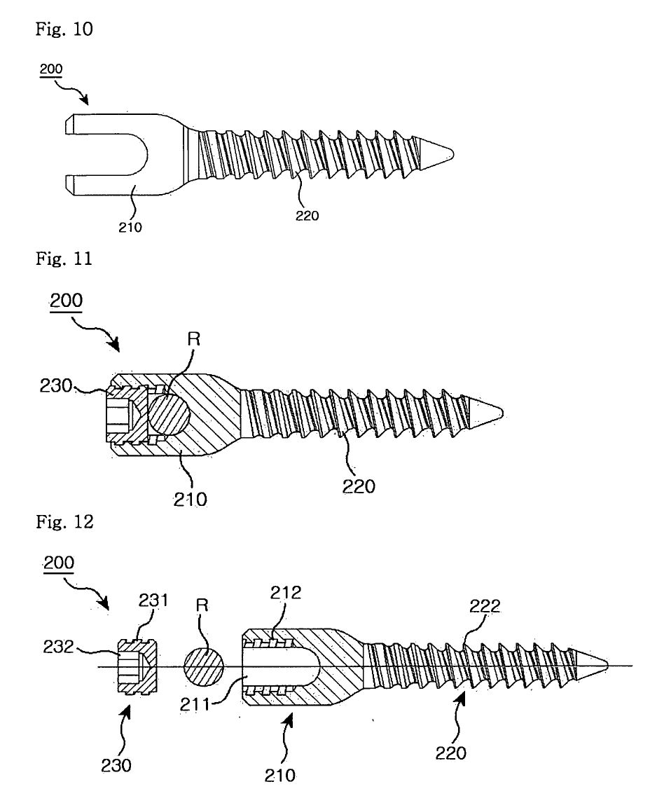

Describing another embodiment of the present invention with reference to FIGS. 10 to 12, the spinal pedicle screw assembly 200 includes a head-coupling element 210 having an upper and a lower end, a screw rod 220 integrally formed with the lower end of the head-coupling element 210, and a tightening screw 230 firmly positioning the spine rod in the head-coupling element 210.

The head-coupling element 210 is shaped like a cup with a rounded bottom, having the U-shaped rod-receiving channel 211 cut through its side wall. The rod-receiving channel 211 is opened towards the upper end, and has a width closely fitting the spine rod R so as to prevent the spine rod R inserted therein from lateral motion. Also provided along the curvature of the inside of the rod-receiving channel are female screw threads 212 tapering towards the periphery of the head-coupling element. In this case, the central axis of the taper is perpendicular to the central axis of the head-coupling element 210, and the cut between the female screw threads is tapered towards the central axis of the head-coupling element 210. The taper serves to compensate for an adverse effect of a vertical external force by maximizing the force of the head-coupling element engaging the tightening screw 230 with male screw threads 231 corresponding to the female screw threads 212.

The screw rod 220 also has a periphery provided with male screw threads 222, fixedly inserted into a pedicle by means of a separate turning device (not shown). The screw rod 220 is tapered towards its lower end. More specifically, the lower end of the screw rod is cone-shaped to facilitate its insertion into the pedicle, and its diameter becomes largest at the upper end thereof. In addition, the thickness of the male screw threads 222 is gradually increased towards the head-coupling element 210, having maximum at the upper end, so as to maximize the final fixing force of the screw rod 220 inserted into the pedicle.

The male screw threads 231 of the tightening screw 230 are tapered towards the center of the head-coupling element 210 so as to match the female screw threads 212. The tightening screw 230 is preferably provided with a hexagonal or octagonal wrench depression 232 at the top thereof, whereby it is turned by a wrench (not shown), engaged by the rod-receiving channel 211 of the head-coupling element 210 to fix the spine rod R. Thus, the spinal pedicle screw assembly 200 is fixedly inserted into a pedicle by means of a separate turning device engaging the wrench depression 232 of the tightening screw 230, while the spine rod R is tightly interposed between the tightening screw 230 and the bottom of the rod-receiving channel 211. The spine rod R engaged by the spinal pedicle screw assembly 200 is shown in FIG. 11, wherein the uniquely tapered engagement of the female screw threads 212 of the head-coupling element 210 and the male screw threads 231 of the tightening screw 230 compensate for the adverse effect of an external force exerted to the spinal portion implanted with the inventive spinal pedicle screw assembly, thus preventing the spine rod from loosening.

Consequently, the inventive spinal pedicle screw assembly successively endures a large external force exerted to the implanted spinal portion after spinal fixation surgery with the help of the uniquely tapered engagement of the head-coupling element and tightening screw and the strongly resistive contact between the restraining plate and the knurled spherical head of the screw rod, thus significantly improving the recuperation of the patient.

While the present invention has been described in connection with specific embodiments accompanied by the attached drawings, it will be readily apparent to those skilled in the art that various changes and modifications may be made thereto without departing the gist of the present invention.

1. A spinal pedicle screw assembly including a spinal pedicle screw cooperating with a spine rod for fixing a spine, comprising:
a head-coupling element having an upper and a lower end, a rod-receiving channel opening toward said upper end for receiving said spine rod, female screw threads formed on the inside of said rod-receiving channel, and a connecting bore of a given size extending through said lower end, said connecting bore having an upper end toward said rod-receiving channel and an opposite lower end;
a screw rod having an upper and a lower end, a spherical head integrally formed with the upper end thereof for being inserted into said connection bore, and male screw threads formed on the periphery of said screw rod between the spherical head and lower end thereof, said screw rod being fixedly inserted into the pedicle by means of the male screw threads thereof;
a tightening screw having a periphery formed with male screw threads for engaging the female screw threads of said rod-receiving channel so as to fix said spine rod in said head-coupling element; and
a final fixing screw threadedly engaged by said connecting bore for preventing said spherical head from disconnecting from said head-coupling element.

2. A spinal pedicle screw assembly as defined in claim 1, further including a restraining plate fixedly positioned between the upper end of said connecting bore and said spherical head for restraining said spherical head from moving.

3. A spinal pedicle screw assembly as defined in claim 1, wherein the cut between the female screw threads of said rod-receiving channel are tapered towards the central axis of said head-coupling element.

4. A spinal pedicle screw assembly as defined in claim 2, wherein the upper end portion of said connecting bore is cut to form a seat for stopping said restraining plate.

5. A spinal pedicle screw assembly as defined in claim 2, wherein said restraining plate has a lower surface provided with a head receiving depression matching the curvature of said spherical head.

6. A spinal pedicle screw assembly as defined in claim 1, wherein said spherical head has a flat top.

7. A spinal pedicle screw assembly as defined in claim 6, wherein said flat top is provided with a central wrench depression of a given shape.

8. A spinal pedicle screw assembly as defined in claim 1, wherein the surface of said spherical head is knurled.

9. A spinal pedicle screw assembly as defined in claim 1, wherein the thickness of the male screw threads formed on the periphery of said screw rod is gradually increased towards said spherical head.

10. A spinal pedicle screw assembly as defined in claim 1, wherein said tightening screw has a top provided with a central wrench depression of a given form.

11. A spinal pedicle screw assembly as defined in claim 1, wherein said final fixing screw has a central through hole for receiving said spherical head, the inside of said central through hole being provided with a lower part shaped to match the curvature of said spherical head.

12. A spinal pedicle screw assembly including a spinal pedicle screw cooperating with a spine rod for fixing a spine, comprising:
a head-coupling element having an upper and a lower end, a rod-receiving channel opening toward said upper end for receiving said spine rod, and female screw threads formed on the inside of said rod-receiving channel;
a screw rod integrally formed with the lower end of said head-coupling element and having a periphery provided with male screw threads, said screw rod being fixedly inserted into the pedicle by means of the male screw threads thereof; and
a tightening screw having a periphery formed with male screw threads for engaging the female screw threads of said rod-receiving channel so as to fix said spine rod in said head-coupling element.

13. A spinal pedicle screw assembly as defined in claim 12, wherein the cut between the female screw threads of said rod-receiving channel are tapered towards the central axis of said head-coupling element.

14. A spinal pedicle screw assembly as defined in claim 12, wherein the thickness of the male screw threads formed on the periphery of said screw rod is gradually increased towards said head-coupling element.

15. A spinal pedicle screw assembly as defined in claim 12, wherein said tightening screw has a top provided with a central wrench depression of a given shape.
